(19) United States
(12) Patent Application Publication
(10) Pub. No.: US 2019/0277823 A1
(43) Pub. Date: Sep. 12, 2019

(54) VOLATILE ORGANIC COMPOUNDS FROM NATURAL SPECIMENS AND USES THEREOF

(71) Applicants: ALISON G. SIMON, GREENSBORO, NC (US); KENNETH G. FURTON, HOMESTEAD, FL (US)

(72) Inventors: ALISON G. SIMON, GREENSBORO, NC (US); KENNETH G. FURTON, HOMESTEAD, FL (US)

(73) Assignee: THE FLORIDA INTERNATIONAL UNIVERSITY BOARD OF TRUSTEES, MIAMI, FL (US)

(21) Appl. No.: 16/285,911

(22) Filed: Feb. 26, 2019

Related U.S. Application Data
(60) Provisional application No. 62/640,625, filed on Mar. 9, 2018.

(51) Int. Cl. G01N 33/00; G01N 30/88

(52) U.S. Cl. CPC G01N 33/0098; G01N 30/88; G01N 2333/37; G01N 2560/00; G01N 2030/8854

(57) ABSTRACT

Certain embodiments of the invention pertain to compositions comprising one or more volatile organic compounds identified from a natural specimen. These compositions can be used as mimic training aids to train animals to identify the natural specimen based on odor. Animals so trained can then be used to identify by odor the natural specimen in a natural environment. The mimic training aids disclosed herein provide consistent training aids as well as avoid the use of live organisms, particularly, live fungal spores. Other embodiments of the invention pertain to fractionation techniques to create reliable and comprehensive mimic training aids for animals. Further embodiments of the invention provide methods of identifying a natural specimen in a natural environment by providing an animal trained to identify a natural specimen by odor, commanding the animal to sniff the natural environment, and identifying the natural specimen from the natural environment based on the animal's signal.

### CROSS-REFERENCE TO A RELATED APPLICATION

This application claims the benefit of U.S. provisional application Ser. No. 62/640,625, filed Mar. 9, 2018, which is incorporated herein by reference in its entirety.

### BACKGROUND

Volatile organic compounds (VOCs) are used by canines as a standoff detection method to locate or identify the presence or absence of target substances. Trained detection canines are currently used in many environmental applications, such as locating invasive and threatened or endangered species for tracking or research efforts. However, training aids are a challenge due to the high risk of spreading invasive species or diseases as well as issues related to legality, acquisition, movement, and containment of targets. Generally, training aids are either live species or feathers, egg shells, nests, carcasses, feces, or other related items left behind by the target species. These aids are often difficult to obtain and face issues of decay or microbial action that could cause contamination.

Canine substance detection is a diverse field, including traditional applications such as narcotics, explosives, currency, firearms, human scent, human remains, and ignitable liquid residues as well as more recent medical and environmental applications. Within environmental detection, canines have been used to identify mold, bedbugs, termites, wildlife scat, plants and agricultural products, as well as endangered and invasive species. While canines have been used for the environmental detection of various targets, application in this area is still not widespread, due in part to the lack of affordable and reliable mimic training aids that provide safe, long-lasting, and easily accessible alternatives. For many environmental targets, nonliving training aids can be found, such as scat, nests, burrows, carcasses, or other items left behind by the target species. However, for many targets in wildlife detection, nonliving training aids cannot be utilized and live training aids present high risk. In particular, for plants or pests, such as fungi living training aids are often not possible because of the high risk for spreading the targeted species. Additionally, training a canine using a live target presents several challenges in regards to rarity of the species, legality of obtaining and maintaining the species, methods of acquisition, and possibilities of spreading the species should it escape the containment system. Mimic training aids can be effective in preventing the use of live species as training aids, which may be a biohazard to the canine and/or handler and has the potential risk of spreading the biological pest. The training process typically includes using hazardous or dangerous live substances and is unique to each individual target species and canine trainer, leading to a variety of approaches. Though mimic training aids have not yet been applied to environmental substance detection, canines are nonetheless being trained in this field. Mimic training aids can enhance training by making this process more uniform across the field.

### BRIEF SUMMARY

Certain embodiments of the invention provide compositions comprising one or more VOCs identified from a natural specimen. These compositions mimic the odors of the natural specimen and thus can be used as mimic training aids to train animals, for example, canines, to identify the natural specimen based on odor. For example, certain such mimic training aids can be used to train canines that can then identify an invasive species by odor. Canines so trained can then be used to identify by odor the biological advancement of an invasive species and promote conservation efforts. The mimic training aids disclosed herein provide consistency in the training aids as well as avoid the use of live organisms, particularly, live organisms, such as animals or fungal spores.

Other embodiments of the invention pertain to fractionation techniques to create reliable and comprehensive mimic training aids for animals. Further embodiments of the invention provide methods of identifying a natural specimen in a natural environment by providing an animal trained to identify a natural specimen by odor, commanding the animal to sniff the natural environment, and identifying the natural specimen from the natural environment based on the animals' signal.

### BRIEF DESCRIPTION OF DRAWINGS

FIG. 1 shows a schematic of sample selection in avocado groves. There is a two-row barrier of non-symptomatic trees between infected and uninfected samples.

FIGS. 2A-2C provides a design of canine experiments over three days. (2A) Took place in a mango grove with 6 mph (10 kph) SE winds. (2B) Took place in a longan grove with 10 mph (16 kph) NE winds. (2C) Took place in a mango grove with 15 mph (24 kph) wind gusts in the SW direction.

FIGS. 3A-3H provides various ring closures of farnesene (center molecule) leading to nine identified VOCs in avocado wood samples, where (3A) is the eudesmene backbone; (3B) is the guaiene backbone; (3C) is the germacrene backbone; (4D-4G) is the cadinene, muurolene, & calamenene backbone; and (4H) is the humulene backbone.

FIG. 4 shows chromatograms of fractions A, B, C, D, and W presented to canines. On the chromatograms representing fractions A, B, C, and D, the compounds contained in each vial are highlighted. Compounds are labeled in fraction W: 1) α-cubenene, 2) δ-elemene, 3) copaene, 4) (−)-alloaromadendrene, 5) γ-muurolene, 6) α-selinene, 7) δ-cadinene, 8) calamenene, and 9) caryophyllene oxide.

FIG. 5 is a flow chart of the process of fractionation, verification, and canine trials.

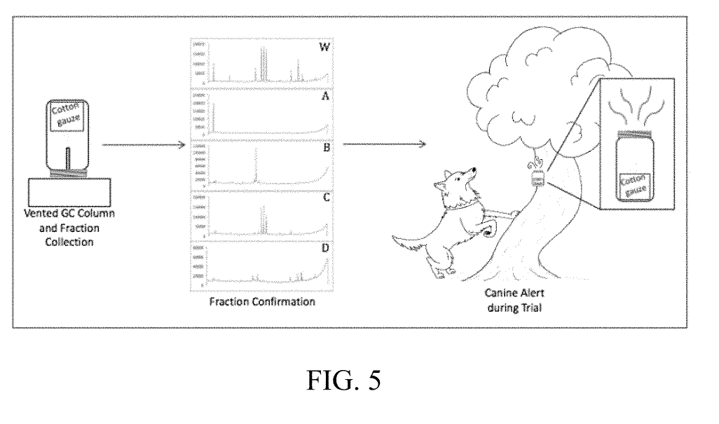

### DETAILED DESCRIPTION

In certain embodiments, the invention provides methods for using gas chromatography to fractionate volatile compounds that form complex odor profiles of a natural specimen. The complex odor profile can comprise headspace volatile compounds from a natural specimen.

The odor profiles so identified can be used in canine training for identifying by odor the source of the volatile compounds, i.e., the natural specimen. Such complex odor profiles of a natural specimen can be used as a mimic training aid to train animals, such as canines, to identify the natural specimen by odor.

Accordingly, an embodiment of the invention provides a method for isolating and identifying volatile compounds from a natural specimen. In one embodiment, the invention provides a method for isolating and identifying headspace volatile compounds from a natural specimen.

Headspace volatile compounds of a natural specimen can be identified by various techniques. In a preferred embodiment, headspace solid phase microextraction-gas chromatography-mass spectrometry (HS-SPME-GC-MS) is used. Additional techniques suitable for identifying headspace volatile compounds are well known in the art and such embodiments are within the purview of the invention.

The term “headspace volatiles of a natural specimen” as used herein refer to volatile organic compounds emitted from the natural specimen. Headspace volatiles can be accumulated through static methods, i.e., accumulated in an enclosed container in which the specimen is placed, or dynamic methods, i.e., purged from the specimen, for example, by application of heat and/or air flow. Depending on the specimen such accumulation can occur within a specified period of time, for example, between 30 and 90 minutes, preferably, between 40 and 80 minutes, even more preferably, between 50 and 70 minutes, and most preferably, about 60 minutes. A skilled artisan can determine an appropriate time for accumulation depending on the specimen and such embodiments are within the purview of the invention.

The natural specimen can be an animal, an animal secretion or excrement, a plant, a plant secretion, a plant pathogen, a microorganism, or a plant or animal tissue infected with a pathogen. A plant pathogen can be a virus, bacterium, a fungus or a protozoan. A microorganism can be a bacterium, a virus, a fungus, or a protozoan. Additional examples of a natural specimen suitable for use according to the instant invention are well known in the art and such embodiments are within the purview of the invention.

Examples of viral infection affecting plants, against which the subject invention is useful, include, but are not limited to Carlavirus, Abutilon, Hordeivirus, Potyvirus, Mastrevirus, Badnavirus, Reoviridae Fijivirus, Oryzavirus, Phytoreovirus, Mycoreovirus, Rymovirus, Tritimovirus, Ipomovirus, Bymovirus, Cucumovirus, Luteovirus, Begomovirtts, Rhabdoviridae, Tospovirus, Comovirus, Sobemovirus, Nepovirus, Tobravirus, Benyvirus, Furovirus, Pecluvirus; Pomovirus; or a mosaic virus, such as alfalfa mosaic virus, beet mosaic virus, cassava mosaic virus, cowpea mosaic virus, cucumber mosaic virus, panicum mosaic satellite virus, plum pox virus, squash mosaic virus, tobacco mosaic virus, tulip breaking virus, or zucchini yellow mosaic virus.

Examples of bacterial infections affecting plants, against which the subject invention is useful, include, but are not limited to, Pseudomonas (e.g., P. savaslanoi, Pseudomonas syringae pathovars); Ralstonia solanacearum; Agrobacterium (e.g., A. tumefaciens); Xanthomonas (e.g., X oryzae pv. oryzae; X campestris pathovars; X axonopodis pathovars); Erwinia (e.g., E. amylovora); Xylella (e.g., X fastidiosa); Dickeya (e.g., D. dadantii and D. solani); Pectobacterium (e.g., P. carotovorum and P. atrosepticum); Clavibacter (e.g., C. michiganensis and C. sepedonicus); Candidatus Liberibacter asiaticus; Pantoea; Ralstonia; Burkholderia; Acidovorax; Streptomyces; Spiroplasma; Phytoplasma; huanglongbing (HLB, citrus greening disease).

An animal can be a nematode, for example, nematodes infecting plants. Examples of nematodes are the cyst forming nematodes of the genus Heterodera (e.g., H glycines, H avenae, and H shachtii) and Globodera (e.g., G. rostochiens and G. pallida); the stubby root nematodes of the genus Trichodorus; the bulb and stem nematodes of the genus Ditylenchus; the golden nematode, Heterodera rostochiensis; the root knot nematodes, of the genus Meloidogyne (e.g., M. javanica, M. hapla, M. arenaria and M. incognita); the root lesion nematodes of the genus Pratylenchus (e.g., P. goodeyi, P. penetrans, P. bractrvurus, P. zeae, P. coffeae, P. bractrvurus, and P. thornei); the citrus nematodes of the genus Tylenchulus, and the sting nematodes of the genus Belonalaimus.

Other plant or crop diseases where the methods and compositions of the instant invention are useful include the pests and/or pathogens causing citrus canker disease, citrus bacterial spot disease, citrus variegated chlorosis, citrus food and root rot, citrus and black spot disease, as well as blights, cankers, rots, wilts, rusts, anthracnose, bacterial spots, club root, corn smut, galls, damping off, downy and powdery mildew, scabs, leaf spot, molds, mosaic virus, leaf blisters, and curls. Further embodiments of the invention provide a composition comprising a substantial portion of the headspace volatiles of a natural specimen. A substantial portion of headspace volatiles contain at least 80% of the compounds present in the headspace volatiles of a specimen. Also, each of the compounds present in a substantial portion of headspace volatiles is present at 20% or more of the relative concentration of the compound compared to the other compounds in the headspace volatiles. For example, if headspace volatiles of a specimen contain 10 compounds, then a substantial portion of the headspace volatiles contain at least 8 of those compounds. Also, if a compound comprises about 5% of the headspace volatiles, then a substantial portion of the headspace volatiles contains at least 1% of that compound.

In certain embodiments, a composition comprising headspace volatiles or a substantial portion of the headspace volatiles of a natural specimen comprises a polymer network, particularly, a sol-gel polymer network, that encapsulates the headspace volatiles or a substantial portion of the headspace volatiles.

Certain examples of sol-gel polymer networks useful for producing the polymer compositions of the invention are described in United States Patent Application Publication No. 2016/0324120, which is incorporated herein in its entirety, particularly, paragraphs [0049] to [0055]. In preferred embodiments, the polymer compositions comprise phenethyltrimethoxysilane (PE-TMS), tetraethyl orthosilicate (TEOS), or tetramethyl orthosilicate (TMOS).

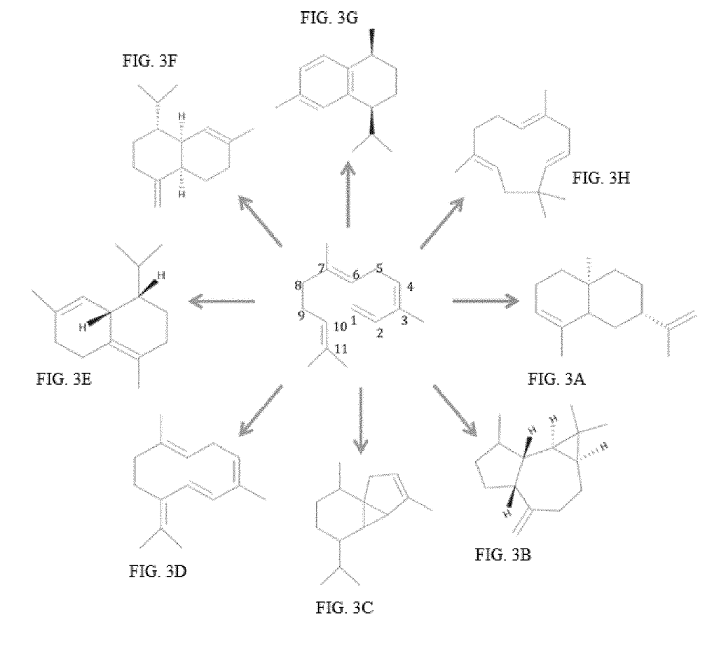

Specific embodiments of the invention provide a sol-gel polymer composition comprising the compounds provided in FIGS. 3A-3H or Table 5.

A further embodiment of the invention provides a method of training an animal to identify by odor a composition comprising headspace volatiles or a substantial portion of the headspace volatiles of a natural specimen. In preferred embodiments, the animal is a canine, though other animals can be used, for example rats or bees.

To train an animal to identify a composition by odor of the composition comprises associating the odor of the composition with a positive experience for the animal and training the animal to provide a signal when the animal encounters the smell of the composition.

For example, the animal can be given a toy that has the odor of the composition. After sufficient training, the animal associates the odor of the composition with its toy and is motivated to identify the smell in its attempt at finding the toy.

In one embodiment, an animal can be given food/treat immediately after the animal identifies the odor of the composition. After sufficient training, the animal associates the odor of the composition with receiving its food/treat and is motivated to identify the odor in its attempt at receiving food/treat.

Additional examples of training an animal, such as a canine, to identify a composition by odor are well known in the art and such embodiments are within the purview of the invention.

An animal so trained can be used to identify by odor the natural specimen based on identification of the headspace volatiles from the natural specimen. In certain embodiments, the natural specimen is identified in a natural environment, for example, a field, a grove, a plantation, or a forest. In preferred embodiments, an animal trained according to the methods of the invention is commanded to sniff a natural environment, and the natural specimen from the natural environment is identified based on the animals' signal. For example, when the animal is a canine, the canine can sit near the natural specimen or bark to indicate the presence of the natural specimen.

### Definitions:

As used herein, the singular forms “a”, “an” and “the” are intended to include the plural forms as well, unless the context clearly indicates otherwise. Further, to the extent that the terms “including”, “includes”, “having”, “has”, “with”, or variants thereof are used in either the detailed description and/or the claims, such terms are intended to be inclusive in a manner similar to the term “comprising.”

The phrases “consisting essentially of” or “consists essentially of” indicate that the claim encompasses embodiments containing the specified materials or steps and those that do not materially affect the basic and novel characteristic(s) of the claim. The phrase “consisting essentially of” as it applies to a composition consisting essentially of a set of compounds that is used for training an animal to identify a natural specimen by odor, particularly, distinguishing between the two natural specimens by odors of the two specimens, the composition can contain additional compounds that do not affect the ability of the animal to identify the natural specimen by odor or to distinguish between the two natural specimens by odors of the two specimens.

### Materials and Methods

### Sampling Protocols

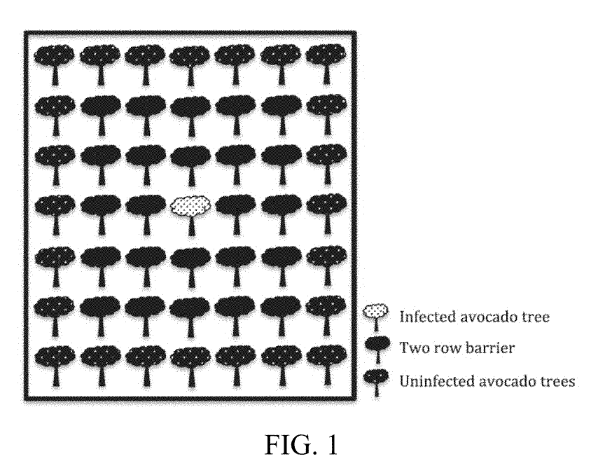

Sampling was focused on studying the VOCs of avocado trees (Persea americana) either uninfected or infected with the laurel wilt disease. Commercial avocado groves in South Florida were sampled with a total of 15 infected and 9 uninfected trees of the Lula variety included in the results presented here. Infected trees were selected if there was visible wilting. Uninfected trees were selected if there was no visible wilting, and there was no wilting in the surrounding two rows of trees (FIG. 1). This method was chosen following the most current recommendation for trenching to isolate infected trees from uninfected areas. Laurel wilt presence in each grove was confirmed through DNA analysis. Location and relevant weather data were recorded for samples collected by the authors, including: infection or no infection, time and day, temperature, humidity, and dew point. Sampling was conducted using a sterilized hatchet. Samples were taken from the trunk of each tree because their volatiles are less variable and their diameters make them the target of X glabratus as well as the host of R. lauricola. Samples of the bark, phloem, and wood were collected from the trunks from between four and five feet high and placed together in 16 oz. sterilized glass Mason jars for transportation to the laboratory. Within 24 h, the samples were taken for analysis using laboratory protocols. Caps were tightened by screwing down all the way then turning back once so as to allow oxygen flow and prevent an anaerobic atmosphere.

### Laboratory Protocol

Laboratory analysis was performed in two separate procedures, each using headspace solid phase microextraction-gas chromatography-mass spectrometry (HS-SPME-GC-MS). The first study identified the VOCs released from Lula variety avocado trees, focusing on the differences in the odor production between healthy and infected trees. The second study created a column vent method using a chromatography column vented to the atmosphere to separate and collect these volatiles on cotton gauze. Once the volatiles were collected, they were verified by HS-SPME-GC-MS and presented to trained detection canines in a series of field trials. The protocols for both studies are presented here.

### Volatile Organic Compound (VOC) Identification

For the first study, all tests were performed in triplicate. Subsamples (3.00 g) containing mixtures of bark, phloem, and xylem were placed in 20 mL clear glass vials with PTFE/silicone septa screw caps (Supelco, Bellefonte, Pa.). The cap was placed on top of the vial and wrapped in Parafilm (Neenah, Wis.) for one hour. Then, the caps were tightened completely for one hour equilibration of the VOCs. Conditioned 50/30 μm Divinylbenzene/Carboxen/Polydimethylsiloxane (DVB/CAR/PDMS) solid phase microextraction fibers (Supelco, Bellefonte, Pa.) were exposed to the headspace above samples for one hour extraction. An extraction and instrument blank was run before each set of triplicate samples. Analytes were desorbed directly from fibers into a Varian 3800 Gas Chromatograph/Saturn 2000 Ion Trap Mass Spectrometer (Walnut Creek, Calif.) injection port for separation and analysis. All chemical compounds were obtained from Sigma-Aldrich (St. Louis, Mo.) except benzaldehyde, which was obtained from Fisher Scientific (Waltham, Mass.). A 0.25 mm ID solgel-wax column (SGE Analytical Science, Pflugerville, Tex.) was used with the GC-MS parameters presented in Tables 1a and 1b.

TABLE 1
(a) The temperature ramp used for the GC-MS analysis. (b) The
parameters for the GC containing the 0.25 mm ID solgel-wax
column. (c) The parameters for the GC containing the 0.53 mm
Inside Diameter (ID) solgel-wax column.
(a)

| Temperature (° C.) | Rate (° C./min) | Hold (min) | Total (min) |
|---|---|---|---|
| 50 | 0.0 | 5.00 | 5.00 |
| 80 | 10.0 | 6.75 | 14.75 |
| 120 | 10.0 | 8.50 | 27.25 |
| 260 | 11.0 | 0.00 | 39.98 |

(b)
Parameters: 0.25 mm ID
solgel wax column

| | |
|---|---|
| Split Ratio: | 10:1 |
| Desorption: | 20 min |
| Low Mass: | 33 m/z |
| High Mass: | 300 m/z |
| Injection Temp: | 250° C. |
| Flow rate: | 1.0 mL/min |

(c)
Parameters: 0.53 mm ID
solgel wax column

| | |
|---|---|
| Split Ratio: | Off |
| Desorption: | 20 min |
| Low Mass: | 33 m/z |
| High Mass: | 300 m/z |
| Injection Temp: | 250° C. |
| Flow rate: | 6.0 mL/min |

### Novel Column Vent Method Development

The second study achieved separation and collection of the VOCs in the headspace above infected samples of avocado trees using a 0.53 mm ID solgel-wax column (SGE Analytical Science, Pflugerville, Tex.) left venting to the atmosphere. Sample collection and SPME methods were followed according to same procedures as above. VOCs were desorbed directly from fibers into Bruker Scion 346-Gas Chromatograph (Billerica, Mass.) injection port for separation (see Table 1c for parameters). Fractions of the odor were collected on U.S.P. Type VII sterile 2″×2″, 8ply cotton gauze (Independent Medical Co-op, Inc., Daytona Beach, Fla.) in 10 mL clear glass vials with PTFE/silicone septa caps (Supelco, Bellefonte, Pa.) according to Table 2.

After a one hour equilibration, SPME fibers were exposed to the headspace of the gauze for a one hour extraction. The fibers were directly desorbed in a Varian 3800 Gas Chromatograph/Saturn 2000 Ion Trap Mass Spectrometer injection port for verification of the VOCs contained in each vial. Note that the only differences between the two GC parameters are the split ratio and flow rate, which had to be altered to ensure the fractions contained accurate separations of the chromatographic data.

TABLE 2
Division of fractions by minute for
column vent procedure

| | Fraction | Fraction end |
|---|---|---|
| Label | begin (min) | (min) |
| A | 0.00 | 10.00 |
| B | 10.01 | 20.00 |
| C | 20.01 | 30.00 |
| D | 30.01 | 39.98 |
| W | 0.00 | 39.98 |

### Canine Field Experiments

Two detection canines trained to detect avocado trees infected with laurel wilt disease were used in this study. The handler-canine teams were certified through the International Forensic Research Institute/National Forensic Science Technology Center (IFRI/NFSTC) Detector Dog Team Certification utilizing the latest best practices by the Scientific Working Group on Dog and Orthogonal detector Guidelines (SWGDOG). Canine 1 (Belgian Malinois) and Canine 2 (Dutch Shepherd) were not previously trained for detection work. They were initially trained using scent association with a universal detector calibrant (patent US9250222).

Canines were then trained for five days per week during two hour sessions. Each dog typically ran two to four trials per training session. Training for laurel wilt disease detection continued for 10 months prior to the study. To verify canine accuracy, trials were done at least biweekly in avocado groves containing infected trees. Training was done according to SWGDOG SC2 General Guidelines and the dogs were housed according to SWGDOG SC4 Kenneling and Healthcare. The canines were all trained to sit at the base of a tree containing the target odors as a positive response.

For laurel wilt detection, training aids were made from a combination of bark, phloem, and xylem obtained from infected avocado trees and heat sealed in controlled odor mimic permeation systems (COMPS) (patent application publication US 2008/0295783) made of 3″×3″ 4 MIL low density polyethylene (LDPE) bags, all stored in the same aluminum bag when not in use. COMPS were used to supply a deployable training aid containing known amounts of odor, providing reproducible and known dissipation rates. It is a simple, disposable, and low cost training aid assembled from permeable polymer containers stored inside non-permeable packaging, which allows pre-equilibration. Advantages of COMPS are that they can be optimized for any desired target odor and the odor contained within is not prone to contamination from outside sources.

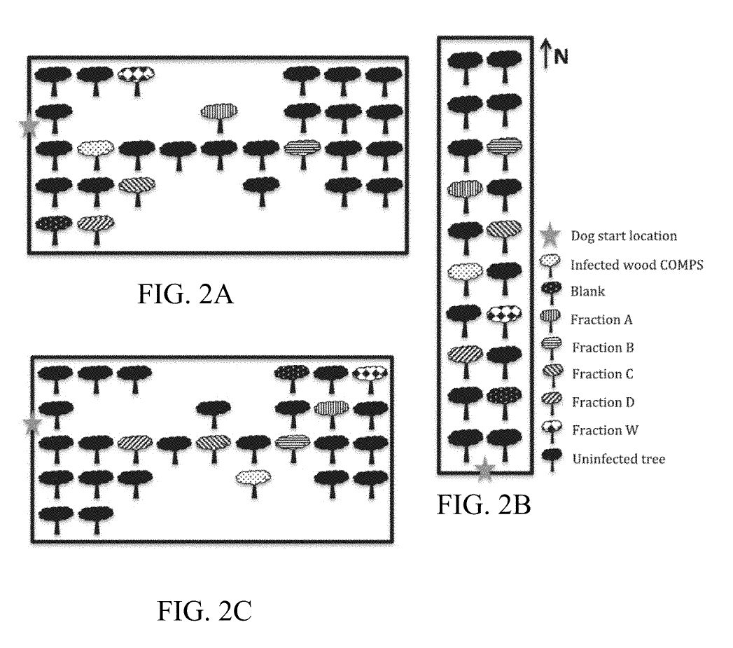

The fractions (A, B, C, D, and W as shown in Table 2) collected from the column vent design were presented to the canines in a series of trials. Additionally, blank cotton gauze in vials and COMPS containing infected avocado wood were used as controls. Trials were performed in a mango (Mangifera indica) grove and a longan (Dimocarpus Tongan) grove to avoid possible introduction of avocado tree odors. The vials were randomly selected and then placed 4-5 feet high in trees using a random number generator. Relevant location and weather data were recorded for each trial, including: time and day, temperature, humidity, and dew point. Three trial days were considered for this study for a total of six canine runs. The canine trial designs are given in FIGS. 2A-2C.

The optimization of the experiment was performed with a 100 ppm standardized solution of eight compounds that elute with retention times across all four fractions. The purpose of this was to ensure the collected fractions were separated properly. The percent loss was calculated for each compound by first injecting 1 μL of 100 ppm liquid solution in the 0.25 mm ID solgel-wax column and then performing the column vent method using the 0.53 mm ID solgel-wax column. These percent losses are given in Table 3. Percent loss for the compound in Fraction A was 43.78%. In Fraction B, the percent loss was 61.71%. For Fraction C, the percent loss ranged from 73.43% to 91.58% and for Fraction D, the percent loss ranged from 94.53% to 96.31%.

TABLE 3
Percent loss between direct liquid injection on 0.25 mm ID solgel-wax
column and SPME of headspace of cotton gauze after collection from
vented 0.53 mm ID solgel-wax column for each compound in a
100 ppm standard solution.

| | | | Percent |
|---|---|---|---|
| CAS No | Compound | RT (min) | Loss (%) |
| 123-92-2 | 1-Butanol, 3-methyl-, acetate | 6.452 | 43.78 |
| 100-52-7 | Benzaldehyde | 18.767 | 61.71 |
| 87-44-5 | Caryophyllene | 20.529 | 73.43 |
| 470-40-6 | Thujopsene | 21.209 | 73.77 |
| 25246-27-9 | Alloaromadendrene | 21.964 | 79.76 |
| 103-45-7 | Acetic acid, 2-phenylethylester | 29.172 | 91.58 |
| 60-12-8 | Phenylethyl alcohol | 31.294 | 94.53 |
| 1139-30-6 | Caryophyllene oxide | 32.276 | 96.31 |

Following are examples which illustrate procedures for practicing the invention. These examples should not be construed as limiting. All percentages are by weight and all solvent mixture proportions arc by volume unless otherwise noted.

### EXAMPLE 1

### VOC Identification

In one embodiment, the invention implements gas chromatography to identify one or more VOCs from Raffaelea lauricola to produce a mimic canine training aid. Canines trained to identify the mimic training aid can be used to identify avocado trees infected with the R. lauricola fungus.

R. lauricola is a newly described nutritional ambrosia fungus that entered the United States in the early 2000s. R. lauricola is believed to be spread through its symbiotic vector, Xyleborus glabratus, or the redbay ambrosia beetle (RAB), which carries the fungus to farms as the vector attempts to find food within host trees. This phytopathogen causes the fatal laurel wilt disease, which is spreading throughout the southeastern United States, most notably in Florida commercial avocado groves. Laurel wilt is a vascular disease that causes trees to die by shutting down the flow of water and nutrients in the xylem. VOCs produced from either R. lauricola or healthy avocado treeshave been previously identified in literature, though none of the volatile signatures identified in these five publications are equivalent to that of the laurel wilt disease in avocado trees. This Example provides odor profiles of infected avocado trees in a comparison to the odor profiles of healthy trees. This Example also characterizes the laurel disease using VOC analyses of infected avocado trees as a proof of concept in creating mimic canine training aids containing a comprehensive representation of VOCs.

Headspace solid phase microextraction-gas chromatography-mass spectrometry (HS-SPME-GC-MS) was used to identify the odors present in avocado trees infected with the R. lauricola. Twenty-eight compounds were detected using this method, with nine present in greater than 80% of samples. The majority of these compounds were not commercially available as standard reference materials, and a canine trial was designed to identify the active odors without the need of pure chemical compounds.

To facilitate the creation of a canine training aid, the VOCs above R. lauricola were separated by venting a 0.53 mm ID solgel-wax gas chromatography column to the atmosphere. Ten minute fractions of the odor profile were collected on cotton gauze in glass vials and presented to the detection canines in a series of field trials. The canines alerted to the VOCs from the vials that correspond to a portion of the chromatogram containing the most volatile species from R. lauricola. This innovative fractionation and collection method can be used to develop reliable and cost effective canine training aids.

VOCs were detected in the headspace above Lula variety infected (n−15) and uninfected (n=9) avocado trees using HS-SPME-GC-MS. Nine compounds present in at least 80% of samples were positively identified and confirmed using either standard mixtures or a mass spectral library (NIST 2000 MS Search 2.0 and AMDIS, Gaithersburg, Md.). These compounds included caryophyllene oxide, δ-elemene, copaene, γ-muurolene, δ-cadinene, calamenene, α-selinene, α-cubenene, and (−)-alloaromadendrene (Table 3). Eight of these compounds were found in uninfected samples. three of which were unique to those samples (a-selinene, α-cubenene, and (−)-alloaromadendrene). Six compounds were found in infected samples, only one of which was unique to those samples (caryophyllene oxide).

The nine VOCs identified were all sesquiterpenes from the mevalonate acid (MVA) pathway, which produces secondary metabolites. They were categorized based on the terpene backbone stemming from different synthases leading to their formation. This was done because categories provide more information about the trees' reactions to infection than the individual compounds. Additionally, only two of the nine compounds have commercially available standards.

Five different backbones were identified: eudesmenes; guaienes; germacrenes; cadinenes, muurolenes, & calamenenes; and humulenes (Tables 4 and 5). These various ring closures identified can be seen in FIGS. 3A-3H. For example, α-copaene, the X glabratus beetle attractant, was detected 100% of infected samples and 89% of uninfected samples. Ring closures of C-1 and C-6 plus C-5 and C-10 on the farnesene molecule lead to δ-cadinene in the cadinenes, muurolenes, & calamenenes category (FIG. 3D). α-Copaene is then formed from a bond between C-2 and C-7 on the 6-cadinene molecule.

TABLE 4
Backbones of compounds present in at least 80% of Lula
variety avocado tree samples

| Backbone | Uninfected | Infected |
|---|---|---|
| Eudesmenes | ✓ | X |
| Guaienes | ✓ | X |
| Germacrenes | ✓ | ✓ |
| Cadinenes, Muurolenes, & Calamenenes | ✓ | ✓ |
| Humulenes | X | ✓ |

TABLE 5
Compounds present in at least 80% of Lula variety avocado tree samples

| CAS | | | | | |
|---|---|---|---|---|---|
| No. | Compound | Backbone | Infected | Uninfected | Structure |
| 1139- 30-6 | Caryophyllene oxide | Humulene | ✓ | X | 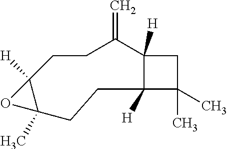 [SMILES: C=C1CC[C@@H]2O[C@]2(C)CC[C@@H]2[C@H]1CC2(C)C] |
| 20307- 84-0 | 6-Elemene | Germacrene | ✓ | ✓ | 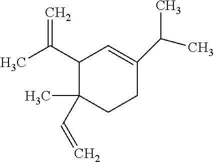 |
| 3856- 25-5 | Copaene | Cadinene, Muurolene, & Calamenene | ✓ | ✓ | 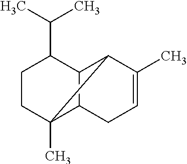 |
| 30021- 74-0 | γ-Muurolene | Cadinene, Muurolene, & Calamenene | ✓ | ✓ | 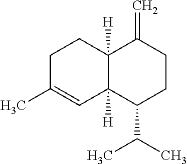 |
| 483- 76-1 | δ-Cadinene | Cadinene, Muurolene, & Calamenene | ✓ | ✓ | 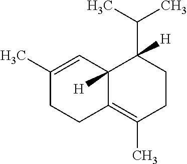 |
| 483- 77-2 | Calamenene | Cadinene, Muurolene, & Calamenene | ✓ | ✓ | 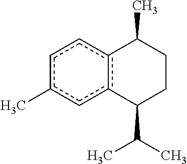 |
| 473- 13-2 | α-Selinene | Eudesmene | X | ✓ | 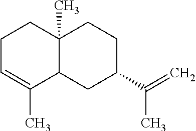 |
| 17699- 14-8 | α-Cubenene | Guaiene | X | ✓ | 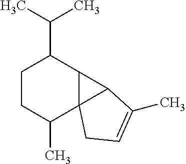 |
| 25246- 27-9 | (−)- Alloaro- madendrene | Guaiene | X | ✓ | 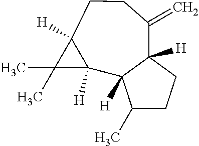 |

### EXAMPLE 2

### Canine Field Experiments

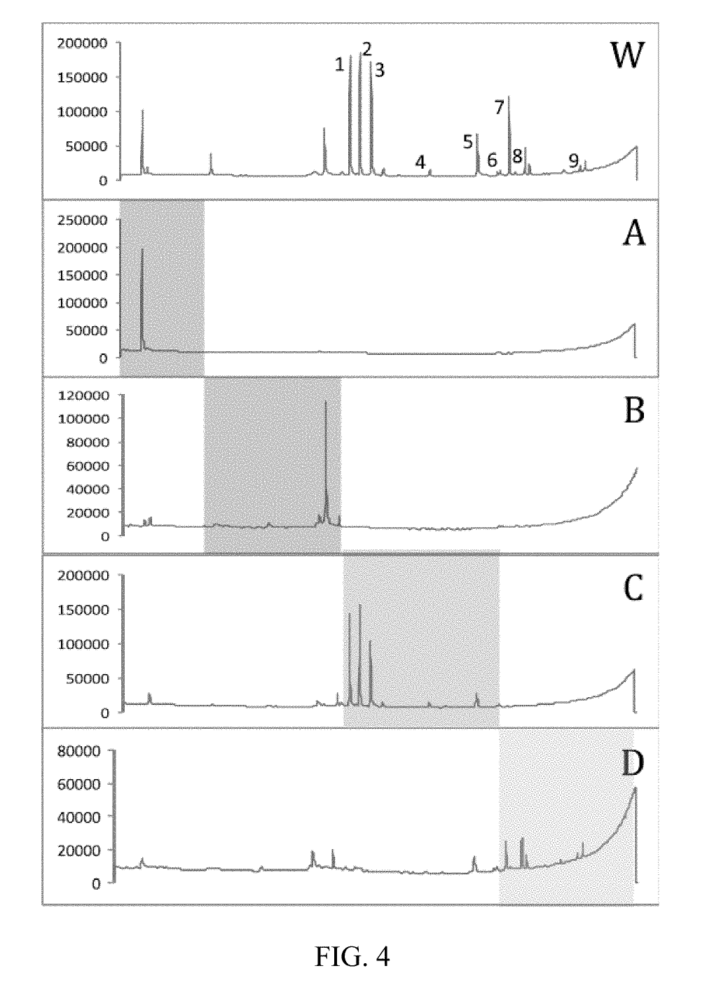

Because only two of the nine compounds are commercially available as standards, the column vent fractions were designed to allow canines to select chromatographic areas of interest from samples of infected avocado trees. The chromatograms of the fractions presented to the canines are displayed in FIG. 4 (refer to Table 3).

The results of the canine trials are presented in Table 6 and the trial designs are presented in FIGS. 2A-2C. Canine trials included in the results are trials where Canines 1 and 2 alerted to the positive control (COMPS) and wind speed was below 15 mph (24 kph). Canines alerted 100.0% to the COMPS with infected wood and 0.0% to the blanks, which were both of the controls. The alert rate for fraction A was 50.0% and the alert rate for fraction B was 33.3%, while the alert rate for fractions C and D were both 0.0%.

TABLE 6
Canine trial results presented as alert
rate (%) and no alert rate (%)

| Training | Alert Rate | No Alert |
|---|---|---|
| Aid | (%) | Rate (%) |
| Blank | 0.0 | 100.0 |
| COMPS | 100.0 | 0.0 |
| W | 100.0 | 0.0 |
| A | 50.0 | 50.0 |
| B | 33.3 | 66.7 |
| C | 0.0 | 100.0 |
| D | 0.0 | 100.0 |

The canines alerted to fractions A and B, which contain the most volatile compounds, demonstrating that the compounds or interest elute in the first half of the chromatogram. Using this information, the compounds from fractions A and B may be used to create training aids that mimic the active odors present in the headspace of infected trees or could be combined in future studies. Multiple training aids can be used to present various ratios of the VOCs from fractions A and B to address the variability of individuals. The canines alerted 100% to fraction W, which represents the complete odor profile, confirming that the active odors of the infected samples' headspace are in fact extracted and collected using the column vent method. The high percent loss for the second half of the chromatogram was determined to not be a factor because the 100% alert rate to fraction W confirmed that all active odors were extracted.

### EXAMPLE 3

### A Novel Method for Extracting and Creating Canine Training Aids

Chemical analysis was performed using headspace solid phase microextraction-gas chromatography-mass spectrometry (HS-SPME-GC-MS). Canine analysis was performed by using the column vent method described to collect odor fractions presented to the canines in a short series of field experiments. This approach allowed the canines to detect the active odors of the laurel wilt disease based on chromatographic areas of interest, which can aid the development of a training method that would not require the use of live cultures, and presents a method that could reduce the dependence on live training aids.

This method helps to overcome the challenge presented by the availability of environmental training aids. Individuals of a species will have different terpenoid mixtures based on genetic, developmental, and environmental factors, though commonalities within species are also prevalent. As presented here, members of the Lula variety of avocados have nine compounds in common in addition to those that are unique to individuals of the species. These compounds are monoterpenes (C10-based compounds) and sesquiterpenes (C15-based compounds) produced from secondary metabolic pathways involved with non-essential life processes, and they mediate direct and indirect plant defense and interactions with fungi such as R. lauricola. Sesquiterpenes are intimately connected with indirect plant defenses against natural enemies, and may illicit defense responses in neighboring, healthy plants as well. These compounds are produced from the mevalonate acid (MVA) pathway, where the condensation of acetate coenzyme A (acetyl-CoA) leads to isopentyl diphosphate (IPP) and its isomer dimethylallyl diphosphate (DMAPP). Farnesyl diphosphate (FDP) synthase combines two molecules of IPP and one of DMAPP to form the C15 diphosphate precursor of sesquiterpenes. Then, the formation of various sesquiterpenes is catalyzed by unique transferases. For example, α-copaene from the cadinenes backbone, the main X. glabratus attractant, is formed from δ-cadinene catalyzed by (+)-δ-cadinene synthase.

Because of the variability of individuals in P. americana, there are several studies that have examined the VOCs produced by healthy avocado trees to try to identify commonalities. Niogret et al. identified six sesquiterpenes common in trunk samples, noting differences among three varieties of avocado trees. Niogret et al. detected 20 sesquiterpenes in trunk samples of avocado trees. Due to proximo-distal gradients, oc-copaene is higher in the trunk than in other areas of the trees and the trunk contains fewer monoterpenes. Niogret et al. identified eleven terepoids, seven of which were sesquiterpenes. These studies are useful in establishing the variability of VOCs in the headspace of avocado trees.

The current study identified nine sesquiterpenes in Lula variety avocado trees, adding to the library of literature and confirming that there is variation between individuals, but that certain commonalities can also be established. This was the first study to identify sesquiterpenes in infected avocado trees.

The compound caryophyllene oxide produced by the humulene backbone was identified as being unique to the infected trees using the parameters described in the methods section. For the five terpene backbones identified, compounds belonging to the groups eudesmene and guaiene decrease in infected trees, suggesting that they may not be necessary for fighting R. lauricola. On the other hand, compounds belonging to the group humulene are increased in infected trees, suggesting that they may be involved in fighting the pathogen. Compounds belonging to the groups germacrene and cadinene, muurolene, & calamenene exist in both uninfected and infected trees, which was expected because copaene, produced from cadinene, is the main attractant for X glabratus.

To overcome the variability distinguishing VOCs of individuals of the species, one approach is to use multiple training aids. In instances where the VOCs present and the threshold of the active odors differ, it can be necessary to use more than one training aid to provide optimal canine training.

A significant current challenge in environmental canine detection is the lack of available training aids containing mimic or pseudo odors of the target odor. However, the identification of the correct active odors and their respective ratios can take years to develop. Canines are generally used to bypass the chemical identification step, but the column vent method presented here and the results described here show how the combination of the analytical HS-SPME-GC-MS method with detection canines can allow for more rapid active odor identification and production of safe training aids. The process involves analytical identification of the compounds present followed by canine identification of chromatographic fractions containing the active odor chemicals.

Analytical instrumentation and canine detection can act as complimentary techniques and result in the rapid identification of active odors in the headspace of target substances. The column vent method permitted a faster process for the identification of VOCs, accelerating the creation of mimic training aids. These training aids can be used as to avoid the risks associated with live training aids, such as rarity of the species, legality of obtaining the species, and methodology of odor or species containment. Additionally, the column vent method can be used to exclude VOCs identified as non-active components, such as fractions C and D in Lula variety infected avocado trees.

These Examples show an application of the invention disclosed herein. Four fractions of laurel wilt-infected avocado trees were presented to trained detection canines, and fractions containing the most volatile compounds were identified as containing the active odorants. The techniques disclosed herein have significant impacts on research and detection methods of environmental targets, leading to a more robust agricultural/environmental defense system and conservation efforts. Mimics for environmental or agricultural threats are not available. This fractionation method can be applied for novel use in the field, changing the way that canine trainers and handlers approach environmental targets. It can be used a training aid or device throughout the training process for detection canines whose target is a complex, evolving headspace. This will allow rapid identification of environmental targets.

All patents, patent applications, provisional applications, and publications referred to or cited herein are incorporated by reference in their entirety, including all figures and tables, to the extent they are not inconsistent with the explicit teachings of this specification.

The examples and embodiments described herein are for illustrative purposes only and various modifications or changes in light thereof will be suggested to persons skilled in the art and are included within the spirit and purview of this application. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

1. A method for isolating and identifying volatile compounds from a natural specimen, comprising isolating and identifying headspace volatiles from the natural specimen.

2. The method of claim 1, wherein said isolating and identifying comprises performing headspace solid phase microextraction-gas chromatography-mass spectrometry (HS-SPME-GC-MS) on the natural specimen.

3. The method of claim 1, wherein the natural specimen is an animal, an animal secretion, an animal excrement, a plant, a plant secretion, a microorganism, or a plant or animal tissue infected with a pathogen.

4. The method of claim 1, wherein the isolating and identifying volatile compounds from the natural specimen comprises separating the headspace volatile compounds by introducing the headspace volatile compounds into a chromatography column.

5. The method of claim 4, wherein the chromatography column is a 0.53 mm inside diameter (ID) gas chromatography column vented to the atmosphere.

6. The method of claim 1, comprising placing the natural specimen and a cotton gauze in a container and collecting the headspace volatiles from the natural specimen on the cotton gauze.

7-10 (canceled)

11. A method of training an animal to identify by odor the composition of claim 7, the method comprising associating the odor of the composition with a positive experience for the animal, and training the animal to provide a signal when the animal encounters the odor of the composition.

12. The method of claim 11, wherein the composition comprises a substantial portion of headspace volatiles of a natural specimen selected from an animal, an animal secretion, an animal excrement, a plant, a plant secretion, a microorganism, or a plant or animal tissue infected with a pathogen.

13. The method of claim 12, wherein the plant tissue infected with the pathogen is a tissue from an avocado tree infected with Raffaelea lauricola.

14. The method of claim 13, wherein the composition consists essentially of the following compounds: caryophyllene oxide, δ-elemene, copaene, γ-muurolene, δ-cadinene. calamenene, α-selinene, α-cubenene, and (−)-alloaromadendrene.

15. A method of identifying a natural specimen in a natural environment, the method comprising providing an animal trained according to the method of claim 11, commanding the animal to sniff the natural environment, and identifying the natural specimen from the natural environment based on the signal provided by the animal.

16. A composition consisting essentially of a substantial portion of headspace volatiles of bark, phloem, xylem, wood, or a combination thereof obtained from an avocado tree infected with Raffaelea lauricola.

17. The composition of claim 16, wherein the substantial portion of the headspace volatiles consists essentially of the following compounds: caryophyllene oxide, δ-elemene, copaene, γ-muurolene, δ-cadinene, calamenene, α-selinene, α-cubenene, and (−)-alloaromadendrene.

18. The composition of claim 17, wherein the substantial portion of the headspace volatiles is encapsulated into a sol-gel polymer network.

19. A method of training a dog to identify by odor the composition of claim 17, the method comprising associating the odor of the composition with a positive experience for the dog, and training the dog to provide a signal when the dog encounters the odor of the composition.

20. A method of identifying an avocado tree infected with Raffaelea lauricola in a natural environment, the method comprising providing a dog trained according to the method of claim 19, commanding the dog to sniff the natural environment, and identifying the avocado tree infected with Raffaelea lauricola in the natural environment based on the signal provided by the dog.

21. The composition of claim 16, wherein the substantial portion of the headspace volatiles consists of the following compounds: caryophyllene oxide, δ-elemene, copaene, γ-muurolene, δ-cadinene, calamenene, α-selinene, α-cubenene, and (−)-alloaromadendrene.

22. The composition of claim 16, wherein the substantial portion of the headspace volatiles is encapsulated into a sol-gel polymer network, and wherein the substantial portion of the headspace volatiles consists of the following compounds: caryophyllene oxide, δ-elemene, copaene, γ-muurolene, δ-cadinene, calamenene, α-selinene, α-cubenene, and (−)-alloaromadendrene.

23. A method of training a dog to identify by odor the composition of claim 22, the method comprising associating the odor of the composition with a positive experience for the dog, and training the dog to provide a signal when the dog encounters the odor of the composition.

24. A method of identifying an avocado tree infected with Raffaelea lauricola in a natural environment, the method comprising providing a dog trained according to the method of claim 23, commanding the dog to sniff the natural environment, and identifying the avocado tree infected with Raffaelea lauricola in the natural environment based on the signal provided by the dog.
